(19) United States
(12) Patent Application Publication
(10) Pub. No.: US 2008/0242878 A1
(43) Pub. Date: Oct. 2, 2008

(54) Semi-Synthetic Taxane Derivatives With Antitumor Activity

(76) Inventors: Gabriele Fontana, Milano (IT); Ezio Bombardelli, Groppello Cairoli (IT); Giovanni Appendino, Novara (IT)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 11/886,253

(22) PCT Filed: Mar. 13, 2006
(86) PCT No.: PCT/EP2006/002285

§ 371 Date: Apr. 28, 2008

(30) Foreign Application Priority Data
Mar. 15, 2005 (IT) MI2005A000415

(51) Int. Cl. C07D 305/08

(52) U.S. Cl. 549/511; 549/510

(57) ABSTRACT

The invention discloses novel secotaxane derivatives of general formula 1, having cytotoxic activity, which can be administered through the injective or oral route, for the therapy of tumors.

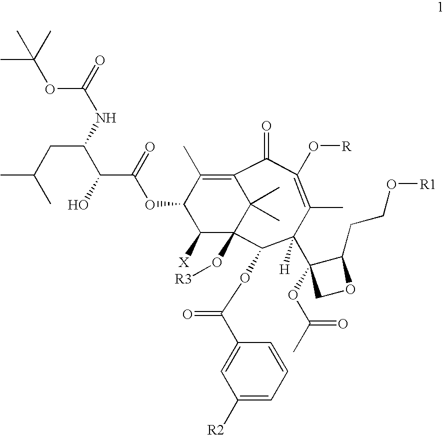

The invention discloses novel secotaxane derivatives having cytotoxic activity.

### BACKGROUND OF THE INVENTION

Taxane-skeleton terpenes, particularly paclitaxel, taxotere and ortataxel, are known to have wide-spectrum antitumor activity. However, the use of these drugs, particularly taxol, involves some drawbacks mainly due to low oral bioavailability, unwanted side effects and quick onset of resistance. For these reasons, the development of novel taxane derivatives having improved oral bioavailability, less side effects and able to avoid the development of resistance, is of high interest.

WO 96/03394 discloses a seco-taxane, known in literature as IDN 5390, (A), characterized by advantageous pharmacokinetic and pharmacodynamic profile

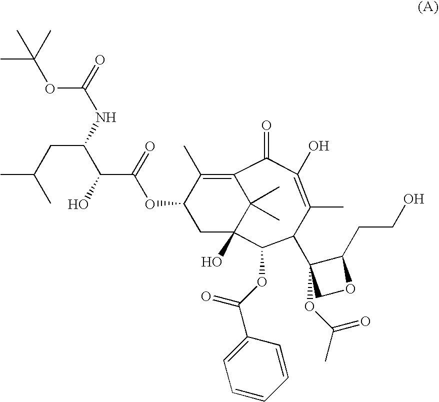

Said molecule exerts a cytotoxic effect related to its ability to inhibit tubulin depolymerization during cell reproduction cycle. Recent studies have illustrated the affinity of the molecule for tubulin I as well as the peculiar specificity for tubulin III whose expression is apparently connected with the onset of resistance. Said characteristics give IDN 5390 therapeutical advantages over taxanes usually employed in medical practice.

### DISCLOSURE OF THE INVENTION

The present invention discloses secotaxane derivatives, analogues of IDN 5390, of general formula 1, having advantageous pharmacokinetic and pharmacodynamic characteristics.

In general formula 1

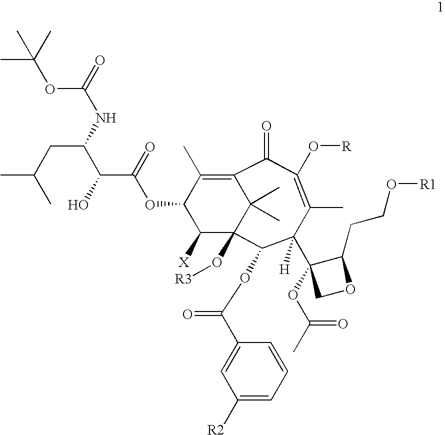

R and R¹, which can be the same or different, are hydrogen, straight or branched C₁-C₆ alkyl, methylthiomethyl or C₁-C₆ acyl, with the proviso that when R is hydrogen or acyl, R¹ cannot be hydrogen or acyl and vice versa;

R² is hydrogen, methoxy, fluorine, chlorine, bromine;

R³ is hydrogen or, together with R⁴, forms a carbonyl or thiocarbonyl group;

X is hydrogen, —OH, —NH₂, —N₃, —OR⁴, NHR⁴.

Taxanes of formula 1 can be prepared directly from IDN 5390 or from compound of formula 2 by functionalization of the 7 and 9 hydroxyls.

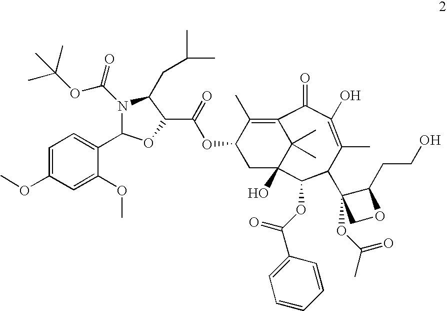

Alternatively, derivatives of formula 1 can be prepared by esterifying novel synthons of formula 3 at the 13-position

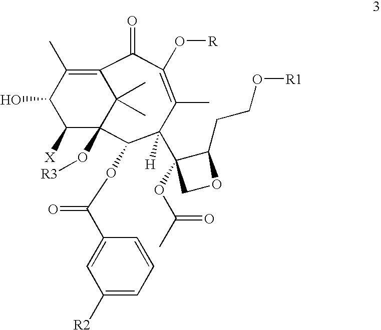

wherein R, R¹, R², R³ and R⁴ are as defined above, with activated derivatives of the acid of formula 4

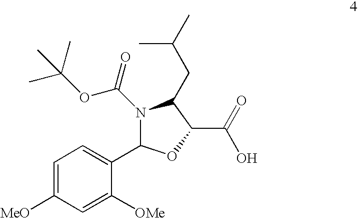

Synthons of formula 3 can be easily obtained from 10-deacetylbaccatin, 14-β-hydroxy-10-deacetylbaccatin or intermediates of formula 5 through reductive fragmentation as disclosed in WO 96/03394, followed by esterification at the 13-position with activated derivatives of acid of formula 4 and introduction of R and R¹ groups.

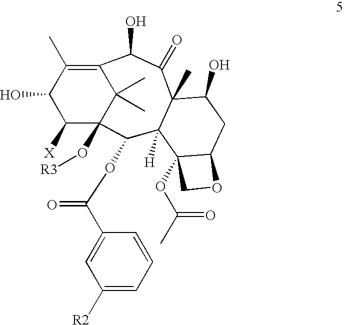

On the other hand, synthons of formula 5 can be prepared by modifying 10-deacetylbaccatin as disclosed in WO 04/024706.

The acid of formula 4 can be obtained as described in WO 01/02407.

The following table shows the cytotoxicity data of some compounds of the invention.

| | | Cytotoxicity (IC₅₀ (nM) | | |
|---|---|---|---|---|
| | | 72 h exposure of cells to | | |
| | | compound ± SE (SRB test) | | |
| Compound | Structure | MCF7 | MCF7/R | RI |
| IDN 5390 | 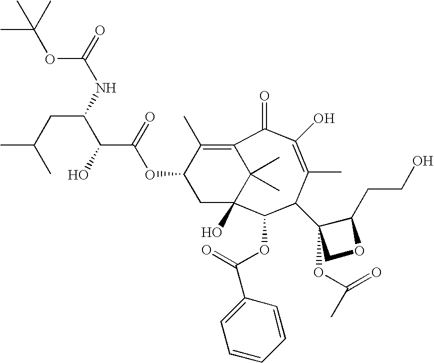 | 11.2 ± 0.81 | 1044 ± 93 | 93 |
| Ex. 2 | 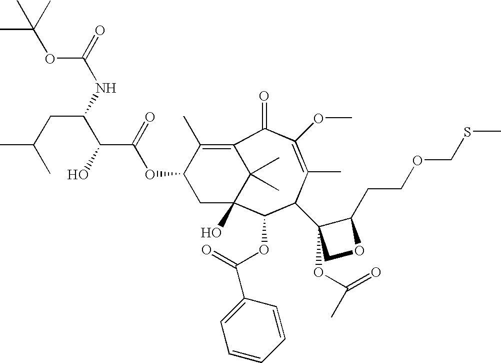 | 103 ± 13 | 558 ± 28 | 5.4 |
| Ex. 3 | 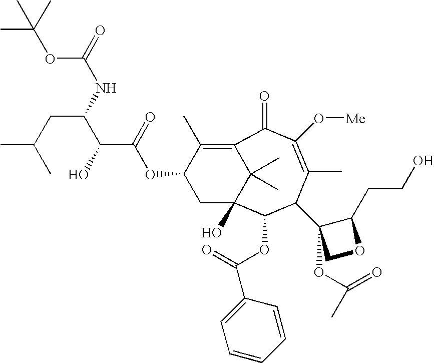 | 38 ± 3.9 | 945 ± 40 | 25 |
| Ex. 4 | 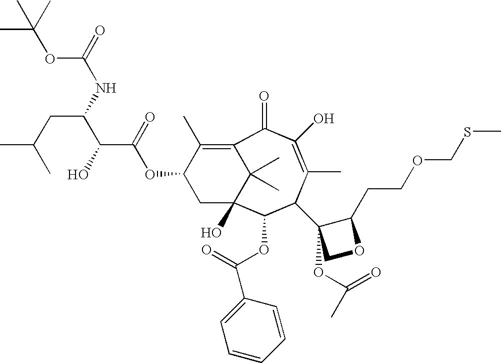 | 26 ± 2.1 | 501 ± 41 | 19 |
| Ex. 5 | 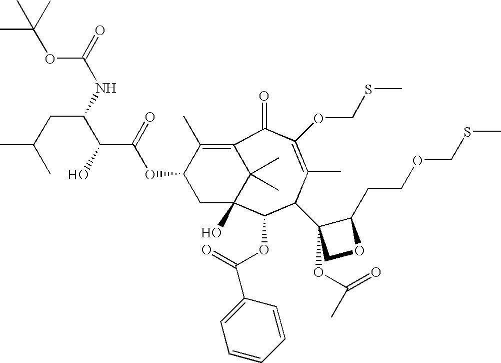 | 58 ± 5.9 | 333 ± 23 | 5.7 |
| Ex. 6 | 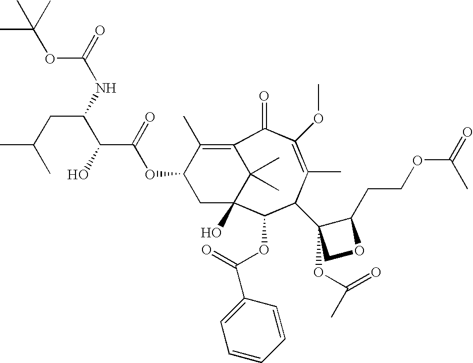 | 66 ± 5.4 | 747 ± 45 | 11 |
| Ex. 7 | 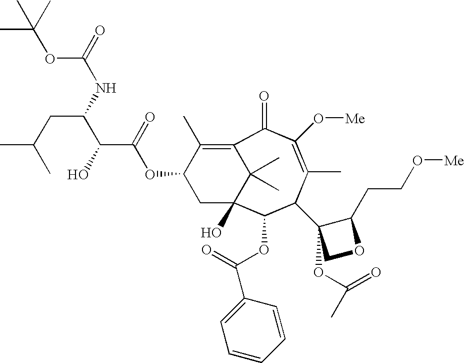 | 25.5 ± 2.6 | 168 ± 9.5 | 6.6 |

It is evident that the derivatives of the present invention have higher cytotoxic activity against resistant cells than the parent compound IDN 5390. Even more importantly, they maintain their activity on wild-type cells and have increased effect on cells of resistant tumors, thereby remarkably lowering cross-resistance index.

The compounds of the invention are suitable for incorporation in conventional pharmaceutical formulations for the parenteral or oral administration, according to the known techniques and in similar or lower dosages than those used known taxanes.

### EXAMPLES

### General Experimental Procedures

IR spectra were recorded on a Shimadzu DR 8001 spectrophotometer.

MS (ESI) was performed on a VG 7070 EQ spectrometer.

¹H- and ¹³C NMR spectra were recorded on Bruker DRX-500 (500 and 125 MHz, respectively) or on Bruker DRX-300 (300 MHz). Solvents signals (CHCl₃/CDCl₃, 7.27/76,9 ppm) were used as internal standard.

Silica gel 60 (70-230 mesh, Merck) was used for open chromatographic columns (CC).

CH₂Cl₂ and triethylamine were dried by distillation from CaH₂.

Organic phases were dried over Na₂SO₄ and evaporated under reduced pressure.

### Example 1

### 13-(N-Boc-N,O-2′,4′-dimethoxybenzylidene-β-isobutylisoserinoyl)-9-O-methyl-C7,8-seco-10-deacetylbaccatin III

100 mg of derivative of formula 2, prepared as disclosed in WO 96/03394, (0.10683 mmoles, MW=936.05) is dissolved in 1 ml of a MeOH/CH₃CN=1:9 mixture. The solution is added with 6 eqmol of 2 M trimethylsilyldiazomethane (TMSCHN₂) in Et₂O (0.6410 mmoles, 320 μl). The reaction is carried out at r.t. and is over after about 7 h with slight formation of 9-methyl-C-secoDAB as a by-product (monitored by TLC-eluent P.E./EtOAc=6:4; Rf_{starting}=0.32; Rf_{product}=0.22; Rf_{side-product}=0.02). The reaction is diluted with AcOEt and acidified with 2 NH₂SO₄. The organic phase is then washed with brine, dried and evaporated.

The crude is purified by column chromatography on a silica (eluent P.E./EtOAc=7:3, then 6:4), thereby obtaining a white powder, mp 165° C.; IR vₘₐₓ (KBr): 3490, 1746, 1719, 1654, 1380, 1318, 1265, 1246 cm⁻¹; ¹H NMR (300 MHz, CDCl₃, 55° C.): δ 8.09 (AA′-Bz), 7.61 (C-Bz), 7.43 (BB′-Bz), 6.14 (br t, J=7.0 Hz, H-13), 5.53 (d, J=9.2 Hz, H-2), 5.67 (d, J=8.0 Hz, H-5), 4.29 (d, J=8.0 Hz, NH), 4.26 (br s, H-20a,b), 3.81 (m, H-7a), 3.76 (s, OMe), 3.67 (m, H-7b), 1.91 (s, OAc), 1.74 (s, H-19), 1.29 (s, BOC).0.95 (d, J=7.4 Hz, H-16, H-19), CI-MS 802 (C₄₂H₅₉NO₁₄)+.

### Example 2

### 13-(N-Boc-β-isobutyl-isoserinoyl)-9-O-methyl-7-O-methylthiomethyl-C7,8-seco-10-deacetylbaccatin III

1.1 g of the compound from Example 1 (1.1578 mmoles; MW=950.07) is dissolved in 8.8 ml of dry DMSO. The solution is added with at room temperature 8.8 ml of Ac₂O and 4.4 ml of glacial AcOH. The reaction is then heated to 35° C. and is complete after approx. 2 h (monitored by TLC-eluent P.E./EtOAc=6:4; Rf_{starting}=0.26; Rf_{product}=0.38). The solution is diluted with AcOEt and neutralized with sat. aq. NaHCO₃. The organic phase is then washed with brine, dried and evaporated. The crude is purified by chromatography on a silica column (eluent P.E./EtOAc=8:2) to afford 934 mg of product (80%).

A solution of 960 mg of the resulting compound (0.9503 mmoles; MW=1010.19) in 9 ml of MeOH is added with 470 μl of a solution of AcCl/MeOH (560 μl in 10 ml of MeOH). The reaction is carried out at room temperature, the mixture is light blue in color; the reaction is complete after approx. 30 minutes (monitored by TLC-eluent dichloromethane/Et₂O=8:2; Rf_{starting}=0.5; Rf_{product}=0.26). The reaction is alkalinised by addition of sat. aq. NaHCO₃ and extracted with AcOEt. The organic phase is then washed with brine, dried and evaporated. The crude is purified by chromatography on a silica column (eluent P.E./EtOAc=8:2 for 47 test-tubes, then 7:3) to afford 704 mg of desired product (86%) as a white powder—IR vₘₐₓ (KBr): 3854, 3676, 2955, 1711, 1661, 1583, 1450, 1272, 1093, 1001, 848, 711 cm⁻¹.—¹H-NMR (300 MHz, CDCl₃, 50° C.): δ 8.01 (d, AA′-Bz), 7.58 (t, C-Bz), 7.48 (t, BB′-Bz), 6.08 (br t, J=7 Hz, H-13), 5.57 (d, J=8 Hz, H-2), 5.21 (m, H-5), 4.65 (d, NH), 4.24 (CH₂—S), 4.16 (H-3 and H-20a), 3.70 (s, —OMe), 2.12 (s, S—CH₃), 1.93 (s, OAc), 1.31 (s, BOC), 1.35 (s, H-17), 1.20 (s, H-16).0.99 (d, H-6′); ESI MS [M+Na]⁺=885 (calc. for C₄₄H₆₃NO₁₄S, 862).

### Example 3

### 13-(N-Boc-β-isobutyl-isoserinoyl)-9-O-methyl-C7,8-seco-10-deacetylbaccatin III

100 mg of IDN 5390 (MW=787.8; 0.127 mmoles) is dissolved in 600 μl of a mixture of MeOH:CH₃CN=1:9.44 μl of Hünig base (0.254 mmoles; 2 eqmol; MW=129; 99%; d=0.742) and 127 μl of a 2.0 M solution of TMSCHN₂ in hexane (MW=114.2; 0.254 mmoles; 2 eqmol; d=0.718) is added under magnetic stirring. The reaction is monitored by TLC, after mini work-up with AcOEt and 2NH₂SO₄ (eluent petrol etere(PE)/EtOAc 6:4, Rf_{IDN 5390}=0.43; Rf_{product}=0.33). After 3 h the reaction is worked up by addition of AcOEt and 2NH₂SO₄, previously dried with brine, then the organic phase is dried over Na₂SO₄ and evaporated in a rotary evaporator. The resulting crude is purified by column chromatography (5 ml of silica) eluting with petroleum ether, petroleum ether/EtOAc 7:3 and 6:4, thereby obtaining 58 mg (57%) of the desired product.

### Example 4

### 13-(N-Boc-β-isobutylisoserinoyl)-7-O-methylthiomethyl-C7,8-seco-10-deacetylbaccatin III

100 mg of the derivative of formula 2 (0.1068 mmoles; MW=936.05) is dissolved in 300 μl of dry DMSO. The solution is added with 215 μl of Ac₂O and 38 μl of AcOH. The reaction is carried out at room temperature and is complete after approx. 20 h (monitored by TLC silica-eluent P.E./EtOAc=7:3; Rf_{starting}=0.12; Rf_{product}=0.29). Acetic acid is neutralized with a NaHCO₃ saturated solution (until disappearance of effervescence) and the mixture is extracted with AcOEt. The organic phase is washed with brine, dried and evaporated. The crude is purified by chromatography on a silica column (eluent P.E./EtOAc=8:2) to afford 75 mg of the protected intermediate (75%).

50 mg of the protected intermediate (0.0502 mmoles; MW=996.17) is dissolved in 1 ml of MeOH. The solution is added with 40 μl of an AcCl/MeOH solution (560 μl in 10 ml of MeOH). The solution turns pale blue almost immediately and the reaction is complete after approx. 50 min (monitored by silica TLC eluent DCM/Et₂O=8:2; Rf_{starting}=0.31; Rf_{product}=0.1). The solution is neutralized with a saturated NaHCO₃ and extracted with AcOEt. The organic phase is washed with brine, dried and evaporated. The crude is purified by chromatography on a silica column (eluent DCM/Et₂O=8:2) to afford 29 mg of desired product (68%) as a white powder—IR vₘₐₓ (KBr): 3798, 3308, 2955, 1713, 1625, 1505, 1367, 1271, 1166, 847, 759, 711 cm⁻¹.—¹H-NMR (300 MHz, CDCl₃, 50° C.): δ 8.00 (d, AA′-Bz), 7.58 (t, C-Bz), 7.49 (t, BB′-Bz), 6.46 (br s, 9-OH), 6.09 (br t, J=8 Hz, H-13), 5.61 (d, J=9 Hz, H-2), 5.11 (d, H-5), 4.69 (d, NH), 4.63 (d, CH₂—S), 4.32 (m, H-20a), 4.17 (m, H-3′), 2.13 (s, S—CH₃), 1.85 (s, OA-c), 1.33 (s, BOC), 1.24 (s, H-16).0.97 (s, H-6′).0.88 (s, H-7′); ESI MS [M+Na]+=871 (calc. for C₄₃H₆₁NO₁₄S, 848).

### Example 5

### 13-(N-Boc-β-isobutylisoserinoyl)-7,9-O-bis(methylthiomethyl)-C7,8-seco-10-deacetylbaccatin III

50 mg of intermediate of formula 2 (0.0534 mmoles; MW=936.05) is dissolved in 450 μl of dry DMSO. The solution is added with 450 μl of Ac₂O and 225 μl of AcOH. The reaction is carried out at room temperature and is complete after approx. 48 h (monitored by TLC silica-eluent DCM./Et₂O=8:2; Rf_{starting}=0.27; Rf_{product}=0.62). Acetic acid is neutralized with sat. NaHCO₃ (until disappearance of effervescence) and the mixture is extracted with AcOEt. The organic phase is washed with brine, dried and evaporated. The crude is purified by chromatography on a silica column (eluent P.E./EtOAc=7:3) to afford 28 mg of intermediate (49%).

155 mg of the resulting compound (0.1467 mmoles; MW=1056.29) is dissolved in 1.5 ml of MeOH. The solution is added with 350 μl of a solution of AcCl/MeOH (560 μl in 10 ml of MeOH). The solution turns pale blue almost immediately and the reaction is complete after approx. 2 h (monitored by TLC on alumine; eluent P.E./EtOAc=7:3; Rf_{starting}=052; Rf_{product}=0.37). The solution is neutralized with sat. NaHCO₃ saturated solution and extracted with AcOEt. The organic phase is washed with brine, dried and evaporated. The crude is purified by chromatography on a silica column (eluent P.E./EtOAc=8:2) to afford 72 mg of desired product (54%) as a white powder—IR vₘₐₓ (KBr): 3801, 3676, 2955, 2360, 1735, 1625, 1507, 1367, 1266, 1165, 848, 711 cm⁻¹.—¹H-NMR (300 MHz, CDCl₃, 50° C.): δ 7.96 (d, AA′-Bz), 7.62 (t, C-Bz), 7.48 (t, BB′-Bz), 6.06 (br t, J=8 Hz, H-13), 5.91 (d, H-2), 5.50 (d, H-5), 5.17 (d, H-20a), 5.85 (d, NH), 4.69 (d, 7-CH₂—S and 9-CH₂—S), 4.35 (m, H-3), 2.17 (s, S—CH₃), 2.08 (s, S—CH₃), 1.95 (s, OA-c), 1.33 (s, BOC).0.97 (s, H-6′ and H-7′); ESI MS [M+Na]⁺=931 (calc. for C₄₅H₆₅NO₁₄S₂, 908).

### Example 6

### 13-(N-Boc-β-isobutylisoserinoyl)-9-O-methyl-7-O-acetyl-C7,8-seco-10-deacetylbaccatin III

A solution of intermediate of formula 2 (100 mg; 0.1068 mmoles, MW=936.05) in 1 ml of a MeOH/CH₃CN=1:9 mixture is added with 6 eqmol of 2 M TMSCHN₂ in Et₂O (0.6410 mmoles, 320 (1). After 7 h at r.t. the reaction is diluted with AcOEt and acidified with 2 NH₂SO₄. The organic phase is then washed with brine, dried and evaporated. The crude is purified by chromatography over C.C. (5 ml of SiO₂, eluent P.E./EtOAc=7:3) to obtain 71 mg of product (70%) (control TLC-eluent P.E./EtOAc=6:4; Rf_{starting}=0.32; Rf_{product}=0.22; Rf_{secODAB}=0.02).

400 mg of the resulting intermediate (0.421 mmoles; MW=950.07) is dissolved in 4 ml of pyridine. The solution is added with 5 eqmol of acetic anhydride (2.105 mmoles; MW=102.09; 198 μl. The reaction is carried out at r.t., after 3 h is complete (monitored by TLC silica-eluent P.E./EtOAc=6:4; Rf_{starting}=0.31; Rf_{product}=0.40) and worked up by addition of 2 NH₂SO₄ and extraction with AcOEt. The organic phase is then washed with brine, dried and evaporated. The crude is dissolved in 4 ml of MeOH and the solution is added with 250 μl of a solution of 560 μl of μl in 10 ml of MeOH. After 25 min. the reaction is worked up by addition of a NaHCO₃ saturated solution and extraction with AcOEt (monitored by TLC on alumina; eluent P.E./EtOAc=6:4; Rf_{starting}=0.66; Rf_{product}=0.26).

The crude is purified by chromatography on a silica column (eluent P.E./EtOAc=7:3 for 60 test-tubes, then 5:5) to afford 242 mg of the desired compound (68% 2 steps) as a white powder.—IR vₘₐₓ (KBr): 3475, 2961, 1742, 1711, 1655, 1385, 1368, 1273, 1250, 712 cm⁻¹.—¹H-NMR (300 MHz, CDCl₃, 50° C.): δ 8.06 (d, AA′-Bz), 7.59 (t, C-Bz), 7.46 (t, BB′-Bz), 6.18 (br t, J=7 Hz, H-13), 5.59 (d, J=9 Hz, H-2), 5.65 (m, H-5), 4.24 (m, NH, H-20a, H-20b), 3.72 (s, —OMe), 2.44 (m, H-6a), 1.86 (s, OAc), 1.29 (s, BOC).0.95 (m, H-16, H-19); ESI MS [M+Na]⁺=867 (calc. for C₄₄H₆₁NO₁₅, 844).

### Example 7

### 13-(N-Boc-β-isobutylisoserinoyl)-7,9-of-O-methyl-C7,8-seco-10-deacetylbaccatin III

1 g of the compound from Example 2 (1.16 mmoles; MW=862.04) is dissolved in 35 ml of 96° EtOH. The solution is added with about 22 g of Raney Ni previously washed once with water and 4 times with EtOH. The reaction is left under H₂ and strong magnetic stirring for 3 h (monitored by TLC-eluent P.E./EtOAc=7:3; Rf_{starting}=0.36; Rf_{product}=0.32). The solution is filtered through Celite and evaporated.

The crude is purified by chromatography on a silica column (eluent P.E./EtOAc=8:2 for 51 test-tubes, then 6:4) to afford 660 mg of the desired product (70%) as a white powder —IR vₘₐₓ (KBr): 3850, 3673, 2955, 2359, 1711, 1659, 1505, 1365, 1272, 1096, 937, 897, 711 cm⁻¹.—¹H-NMR (300 MHz, CDCl₃, 50° C.): δ 8.01 (d, AA′-Bz), 7.58 (t, C-Bz), 7.46 (t, BB′-Bz), 6.08 (br t, J=7 Hz, H-13), 5.60 (d, J=8 Hz, H-2), 5.08 (d, J=10 Hz, H-5), 4.74 (d, NH), 4.27 (m, H-3, H-20a, H-7a, H-7b), 3.73 (s, OMe), 3.40 (s, OMe), 2.50 (m, H-6a and H-14b), 1.92 (s, —OAc), 1.31 (s, BOC), 1.35 (s, H-17), 1.20 (s, H-16).0.99 (d, H-6′), 0.97 (s, H-7′); ESI MS [M+Na]⁺=839 (calc. for C₄₃H₆₁NO₁₄, 816).

1. Compounds of formula 1 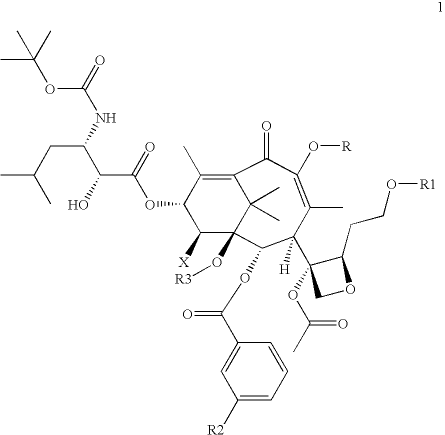
in which:
R and R¹, which can be the same or different, are hydrogen, straight or branched C₁-C₆ alkyl, methylthiomethyl or C₁-C₆ acyl, with the proviso that when R is hydrogen or acyl, R¹ cannot be hydrogen or acyl and vice versa;
R² is hydrogen, methoxy, fluorine, chlorine, bromine;
R³ is hydrogen or, together with R⁴, forms a carbonyl or thiocarbonyl group;
X is hydrogen, —OH, —NH₂, —N₃, —OR⁴, NHR⁴.

2. Pharmaceutical compositions containing the taxanes of claim 1.

3. The use of the taxanes of claim 1 for the preparation of antitumor drugs.

4. Synthons of formula 3 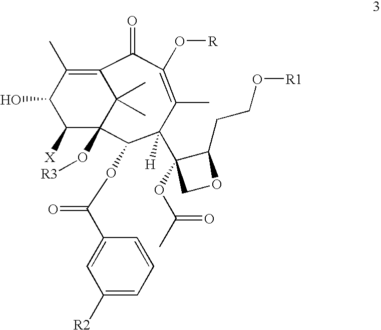
wherein R, R¹, R², R³ and R⁴ are defined as in claim 1.
